(19) United States
(12) Patent Application Publication
(10) Pub. No.: US 2003/0157201 A1
(43) Pub. Date: Aug. 21, 2003

(54) Composition for improving mental capabilities in mammals

(76) Inventors: Maharaj Krishen Pandita, New Delhi (IN); Govind Prashad Dubey, Uttar Pradesh (IN)

Correspondence Address: NEEDLE & ROSENBERG P C, 127 PEACHTREE STREET N E, ATLANTA, GA, 30303-1811, US

(21) Appl. No.: 10/346,290

(22) Filed: Jan. 16, 2003

Related U.S. Application Data
(63) Continuation of application No. 09/554,026, filed on Oct. 27, 2000, now abandoned.
Application No. 09/554,026 is a 371 of international application No. PCT/IN99/00036, filed on Aug. 9, 1999.

(30) Foreign Application Priority Data
Feb. 10, 1999 (IN) 222/DEL/99
Feb. 10, 1999 (IN) 221/DEL/99
Feb. 10, 1999 (IN) 220/DEL/99
Jan. 25, 1999 (IN) 146/DEL/99
Sep. 7, 1998 (IN) 2660/DEL/98
Sep. 7, 1998 (IN) 2658/DEL98

(51) Int. Cl. A61K 35/78

(52) U.S. Cl. 424/725; 514/100

(57) ABSTRACT

The present invention relates to a highly potent herbal composition for improving overall mental performance in children, adults and mentally deficient people characterized by using extracted active plant materials from Brahmi herb and at least one of the herbs from Acorus calamus and Celestrus paniculatus oil.

This is a continuation of 35 U.S.C. § 371 national phase application filed May 8, 2002 from International Application No. PCT/IN99/00036, filed on 9 Aug. 1999, and claiming priority to foreign applications 2658/Del/98, filed 7 Sep. 1998, 2660/Del/98, filed 7 Sep. 1998, 146/Del/99, filed 25 Jan. 1999, 220/Del/99, filed 10 Feb. 1999, 221/Del/99, filed 10 Feb. 1999, and 222/Del/99, filed 10 Feb. 1999, all of which are herein incorporated by reference.

### FIELD OF THE INVENTION

This invention relates to a composition for improving mental capabilities in children, adults and mentally deficient people. In particular though not limited thereto, the composition of the present invention is advantageously used for improving short term memory, long term memory and attention span, by exerting its effect on increasing concentration ability, increasing short term and long term memory span, improving learning ability, speech & recall defects and improving overall mental performance.

### BACKGROUND

It is generally known that Brahmi herb has memory enhancement activity. Brahmi Herb, as used herein, refers to an herbal composition consisting of the whole plants, plant parts, or extracts of Bacopa monnieri and/or Centella asiaticia, both of which can be found growing in wet climates throughout the tropical zones of India. Highest yield was found in valley of Brahmaputra, Assam and West Bengal. It is also known that Bacopa monnieri/Centella asiatica can be processed to obtain either a hydro, hydro-alcoholic or alcoholic extract, and that the herb itself or any of the above extracts may be used for memory enhancement. However, a disadvantage associated with the use of above extracts or the herb is that it contains a substantial quantity of with the use of above extracts or the herb is that it contains a substantial quantity of bitter and other undesirable constituents producing negative effect like presence of alkaloids (inhibition of tissue respiration and alteration of blood pressure), sterols (suppresses the immune competence), raisins (alters mortality) and in case of herbs large quantities are required for achieving the benefits. The herbs also contain varying quantities of active principles depending on various parameters i.e. location, climate etc. Thus, not only making the dose administration impossible but also makes the evaluation of its therapeutic potential difficult.

Extract of Centella asiatica are also known to be used for addressing memory enhancement by improving psychotropic potential.

Herb and herbal extracts of Acorus calamus are also known in the art for memory enhancement. However, such extracts known in the art contain β-asarone present in an amount of about 500-700 ppm. A disadvantage associated with the known Acorus calamus is the presence of β-asarone, which has carcinogenic properties. Acorus calamus is found in Kashmir, Punjab, Himachal Pradesh and Madhya Pradesh.

Celestrus paniculatus (wild) oil is used for reduction in hyper activity of the brain thus permits the nerves to retain the information process for improving the memory.

Memory is a complex process regulated by various factors viz. anxiety, reaction, derangement of neuron transmitters, particularly acetylcholine including serotonin are responsible of the regulation of memory.

Malnutrition, particularly, iodine deficiency, protein deficiency also play a significant role in memory regulation.

Up to this date, nobody has done any work on the combined effect of the above herbs, namely, Brahmi (Bacopa monnieri and Centella asiatica), Acorus calamus and Celestrus paniculatus oil, for improving overall mental performance.

Each of the above mentioned herbs up to now were used individually to increase the memory in general i.e. without any specific indications. No study was carried out ever or published to assess the mode of function of each of the above stated herbs. As a result, these were not having appropriate effect depending upon the lack of particular facet in a particular person whose memory is weak. For example, a child having lower memory due to higher level of anxiety, aggressiveness, Brahmi fails to have any noticeable effect on the memory of the child. Similarly, the properties of Acorus calamus are to increase the protein synthesis thus increases the capabilities of the neuron to retain/increase the information/retention span. The inventor is the first to establish conclusively that memory failure may not be treated, or enhanced appropriately by administering any single herb. As a result of these findings, this invention provides herbal compositions which can now be administered to successfully modulate memory failure due to a number of different causes.

The object of this invention is therefore, to propose a highly potent composition for improving overall mental performance i.e. long term memory, short term memory, attention span, its retention, recall and speech defects by arresting the loss of acetylcholine, increasing capabilities of neurons to retain information, decreasing the hyper excitability of nervous system in the brain by reducing catecholamine level, reducing emotional fluctuation by regulating 5 HT in the brain.

The second object of this invention is to prevent the presence of β-asarone, in quantities of >5 ppm in Acorus calamus, which makes the composition safer and free from side effects.

### THE INVENTION AND ITS DETAILED DESCRIPTION

To achieve the said objectives this invention provides a highly potent herbal composition for improving overall mental performance in children, adults and mentally deficient people characterized by using extracted active plant materials from Brahmi herb and at least one of the herbs from Acorus calanius and Celestrus paniculatus oil.

The said highly potent herbal composition for improving overall mental performance in children, adults and mentally deficient people characterized by using extracted active plant materials from Brahmi herb and at least one of Acorus calamus and Celestrus paniculatus oil in the amounts as given below:

| | |
|---|---|
| Brahmi herb | about 100-500 mg |
| Acorus calamus | about 7.5-35 mg |
| Celestrus paniculatus oil | about 0.02-0.2 ml |

in a total volume of 10 ml or total weight of 10 mg, and the balance amount, if any, is from known additives selected from stabilizers, anti-caking agents, flavoring agents, sweetening agents, thickening agents, preservatives and coloring agents.

This composition will have excellent properties of improving the overall mental performance, for instance, a child having lower memory due to higher level of anxiety and aggressiveness, the Centella asiatica in the composition will decrease serotonin and thereby reduce the anxiety and Celestrus paniculatus oil in the composition will decrease the catecholamine and thereby reduces the aggressiveness and simultaneously making the other components of the composition active and improve the overall mental performance.

The herbal extracts are in the form of a powder, liquid or paste and the quantities used are based on dry powder as given in the composition.

Brahmi herb extract is either obtained from Bacopa monnieri or from Centella asiatica or from combination of both.

The preferred range of the following herbal material is as follows:

| | |
|---|---|
| Bacopa monnieri | about 150-300 mg |
| Centella asiatica | about 25-200 mg |
| Acorus calamus | about 8-20 mg |
| Celestrus paniculatus oil | about 0.04-0.15 ml |

The Bacopa monnieri and Centella asiatica used in the composition has preferably saponins content about 15-25%.

The Acorus calamus used has preferably about 40-55% glycosides and β-asarone content below 5 ppm.

Bacopa monnieri extract and Centella asiatica are hydro-alcoholic organic extracts.

The said Acorus calamus extract is hydro-alcoholic extract of the Rhizome of the herb and the extraction technique is adopted to either eliminate the β-asarone for obtaining extract of Acorus calamus or with β-asarone less than 5 ppm and the contents of β-asarone in the extract is measured by gas chromatography recommended by FDA, USA.

The Celestrus paniculatus is an oil.

The extract of Bacopa monnieri for use in the composition is either prepared by any known process or by refluxing an alcoholic extract of Bacopa monnieri, concentrating to a solid content of about 50-70% and drying the refluxed material at temperature about 50-90° C. under vacuum, treating the dried material with organic solvent preferably chloroform for removal of the soluble fraction and then subjecting the insoluble fraction to refining to obtain a debitterized rich fraction of glycosides.

The said composition is in a powder, tablet, capsule, syrup or in suspension form and also is delivered through any other known carriers such as biscuits/snacks.

In case of composition in the form of tablets, capsules or powder form the Celestrus paniculatus oil is first adsorbed in Aerosol or any other suitable known adsorbing media and used accordingly.

It is generally believed that the Nootropic components present in the composition improves short term memory, long term memory and attention span, by exerting its effect on increasing concentration ability, increasing memory span, improving learning ability and overall mental performance.

Reference is now made to the presence of Centella asiatica for psychotropic effects in the composition, which is responsible for improving the memory facets such as short term, long term memory and concentration ability. It is known that Bacopa monnieri effectively improves such facets and accordingly it is believed that the presence of Centella asiatica is complimentary to that of Bacopa monnieri in improving such mental facets. As both Centella asiatica and Bacopa monnieri are complimentary to each other, the activity of each of such constituents when present in combination has been ascertained and tests have shown that better levels of enhancement of memory facets are obtained where both the constituents are present in the composition. The nootropic & psychotropic properties are achieved. Simultaneously and depending upon the degree of enhancement required of such facets only one of such constituent may be used. Accordingly, and in accordance with one embodiment of this invention, the composition includes only Bacopa monnieri. In accordance with another embodiment and wherein higher all round levels of enhancement of memory is required, the composition contains both Bacopa monnieri and Centella asiatica.

The extract of Shatparvika consists of Acorus calamus and should have glycosides present in the range of about 35 to 65%. Preferably, but without implying any limitation to the scope of the invention, about 40-55% of glycosides is present in the Acorus calamus . Further, β-asarone present in Acorus calamus should not exceed to more than 5 ppm. Acorus calamus is responsible for recall and expression of the stored information from the brain. The extract is hydro-alcoholic extract of the Rhizome of the herb Acorus calamus and the extraction technique is adopted to either eliminate the β-asarone for obtaining extract of Acorus calamus or with β-asarone <5 ppm. The contents of β-asarone in the extract is measured by gas chromatography recommended by FDA, USA.

Reference is now made to the presence of Shatparvika or Acorus calamus in the composition. As described herein above, the function of Shatparvika is to improve the recall and expression properties. However, the composition need not contain Shatparvika in the instance where such properties are not required.

The extract of Vadangul used in the present invention consists of Celestrus paniculatus oil.

To every 10 ml. of the composition in syrup form, the composition contains about 100-500 mg of Bacopa monnieri and preferably between 150-300 mg. If less than 150 mg is present the efficacy of the composition is adversely affected, and desired results can not be obtained. If the composition contains more than 300 mg no additional activity is achieved. In fact, if the composition contains excessively high amount of Bacopa monnieri, such as more than 2000 mg the gastric irritation has been observed.

To every 10 ml of the composition, about 10-500 mg of Centella asiatica is present. Preferably, the composition contains about 25-200 mg of Centella asiatica. Such a composition also contains about 7.5 - 25 mg of Acorus calamus and preferably

The composition may be in a powder, tablet, capsule, syrup or suspension form. Preferably, an alcoholic or hydro-alcoholic extract of Bacopa monnieri prepared by the process of the present invention and as described subsequently herein is employed in the composition, as the bitter constituents are removed therefrom. A particular and advantageous application is with respect to a syrup form, as a bitter palatable taste ought to be avoided. Even in the instance of the composition being in a powder or tablet form, it is preferred that the hydro-alcoholic extract of Bacopa monnieri of the process of the present invention is used in the composition. However, as a bitter palatable taste may still be tolerated in a powder, capsule and tablet form, the known extract of Bacopa monnieri and Bacopa monnieri with the adequate concentration of active components may also be employed.

As desired, herein above, it is known to obtain a hydro, hydro-alcoholic or alcoholic extract of Bacopa monnieri and Centella asiatica. Though each of the aforesaid extracts do contain glycosides in accordance with the present invention the starting material comprises an alcoholic extract of Bacopa monnieri and Centella asiatica, which is refluxed till the herb is exhausted and so as to obtain an extract having glycosides and other compounds certain of which are bitter and undesirable constituents.

The refluxed material is concentrated to have a solid content of about 50-70% under vacuum conditions and that the operating temperature is between 50-70° C. The concentrated product is dried at a temperature of about 50-90° C. It has been found that if higher temperature is employed it would then destroy or change the nature of palatable and active glycosides contained in the extract. The step of drying may be effected in a drum or tray dryer under vacuum at a temperature of about 50 to 90° C., and preferably about 50-70° C. so as to prevent any thermal damage to the palatable glycosides and to obtain a powdered form of the extract. Alternatively, the step of spraying drying may be effected in a spray dryer. The step of drying is carried out for a period of about 40 to 120 secs and preferably for a period of about 60 to 80 sec. If the step of drying is effected in a tray or drum dryer, the step of milling them follows the step of drying is effected in a tray or drum dryer, the step of milling them follows the step of drying. In the instance of spray drying, the operating temperature should preferably be between 70 to 80° C.

The dried extract is subjected to the step of solubilising in solvents namely chloroform. The extracts are soluble in other solvents rather than chloroform, which are preferably not employed, as certain of the bitter constituents present in the extract are also soluble in such other solvents. Thus the step of solubilising is preferably carried out in chloroform as a solvent under atmospheric condition to obtain a soluble fraction and an insoluble fraction. The soluble fraction is rejected and such a process may be repeated for refinement of the insoluble fraction.

The insoluble fraction is filtered and concentrated at about 50-70° C. under vacuum and then dried also at a temperature not exceeding 70° C.

The dried powder is subjected to the step of refinement, which comprises in a first step of dissolving the said powder in water to obtain a soluble fraction. The soluble fraction is filtered and concentrated to about 50-70% solids under vacuum.

Preferably, the step of refining consists in a second step and wherein the concentrated solids obtained from the first step of refining is dissolved in alcohol to obtain an alcohol soluble fraction and a second insoluble fraction. The alcohol soluble fraction is concentrated under vacuum at a temperature of about 50-70° C. to obtain a concentrated powder to about 50-70% solid of content.

The step of refinement comprises in a third step, wherein the alcohol soluble fraction is dissolved in acetone to obtain a first fraction containing acetone soluble material and a second fraction insoluble in acetone and comprising debitterized glycosidic rich fraction, which is also subjected to the step of concentration and then finally dried at a temperature of about 50 to 70° C. The step of drying may be effected after each step of refinement or only after the third step of refinement.

The invention will now be described with reference to the following examples.

### EXAMPLE I

A 10 ml composition in the form of syrup contained the following active ingredients:

about 120 mg of the alcoholic extract of Bacopa monnieri

about 15 mg of extract of Centella asiatica,

about 15 mg of β-asarone free extract of Acorus calamus,

about 0.03 ml of oil of Celestrus paniculatus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

The investigation showed perceptible beneficial effect but of a lower degree in mild cases of mental deficiency.

Attention span, short and long term memory and overall mental performance showed significant improvement, comparatively of lower magnitude) p<0.05), when compared with the placebo treated group.

p=statistical degree of significance.

### EXAMPLE II

A 10 ml composition in the form of syrup contained the following active ingredients:

about 250 mg of the alcoholic extract of Bacopa monnieri

about 40 mg of extract of Centella asiatica,

about 22 mg of β-asarone free extract of Acorus calamus,

about 0.15 ml of oil of Celestrus paniculatus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

A considerable improvement was noted on the parameters under investigation (p<0.001) on comparison with placebo as well as with example I

When the results of both the serials was compared along with placebo it was observed that optimal results could be obtained by the doses given in example II.

Statistically the results were highly significant in example IInd dosages.

### EXAMPLE III

A 10 ml composition in the form of syrup contained the following active ingredients:

about 250 mg of the alcoholic extract of Bacopa monnieri

about 40 mg of extract of Centella asiatica,

about 22 mg of β-asarone free extract of Acorus calamus,

about 0.15 ml of oil of Celestrus paniculatus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

A considerable improvement was noted on the parameters under investigation (p<0.001) on comparison with placebo as well as with example I

When the results of both the serials was compared along with placebo it was observed that optimal results could be obtained by the doses given in example III. Statistically the results were highly significant in example III dosages.

### EXAMPLE IV

A 10 ml composition in the form of syrup contained the following active ingredients:

about 200 mg of the alcoholic extract of Bacopa monnieri

about 25 mg of β-asarone free extract of Acorus calamus,

about 0.10 ml of Celestrus paniculatus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

The composition exhibited marked improvement in nootropic properties. However, the degree of improvement was lesser than composition II. In the absence of Centella asiatica no psychotropic activity could be identified.

However, the composition was beneficial for short term, long term and improvement in the attention span.

The degree of significance noted was p<0.02.

### EXAMPLE V

A 10 ml composition in the form of syrup contained the following active ingredients:

about 200 mg of the alcoholic extract of Bacopa monnieri

about 25 mg of β-asarone free extract of Acorus calamus,

about 0.10 ml of oil of Celestrus paniculatus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

The composition exhibited marked improvement in nootropic properties. However, the degree of improvement was lesser than composition II. In the absence of Centella asiatica no psychotropic activity could be identified.

However, the composition was beneficial for short term, long term and improvement in the attention span.

The improvements in the recall and speech mechanism were significant.

The degree of significance noted was p<0.02.

### EXAMPLE VI

A 10 ml composition in the form of syrup contained the following active ingredients:

about 200 mg of extract of Centella asiatica,

about 25 mg of β-asarone free extract of Acorus calamus,

about 0.10 ml of Celestrus paniculatus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

This composition was tried without Bacopa monnieri and it was noticed that psychotropic properties were dominant, which resulted in improvements in hyper activity and therefore improvement in the α-activity of EEG was noticed. The improvements in the recall and speech mechanism were significant.

### EXAMPLE VII

10 ml composition in the form of syrup contained the following active ingredients:

about 200 mg of the alcoholic extract of Centella asiatica

about 25 mg of β-asarone free extract of Acorus calamus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

This composition was tried without Bacopa monnieri and it was noticed that psychotropic properties were dominant, which resulted in improvements in hyper activity and therefore improvement in the α-activity of EEG was noticed. The improvements in the recall and speech mechanism were significant.

### EXAMPLE VIII

10 ml composition in the form of syrup contained the following active ingredients:

about 200 mg of the alcoholic extract of Bacopa monnieri

about 40 mg of extract of Centella asiatica,

about 0.10 ml of oil of Celestrus paniculatus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

The above composition was tried without the incorporation of Acorus calamus. After 3 months of therapy, significant increase in nootropic and psychotropic properties were noticed. It was noticed that the recall mechanism was of lower significance as compared to composition I.

### EXAMPLE IX

A 10 ml composition in the form of syrup contained the following active ingredients:

about 200 mg of the alcoholic extract of Bacopa monnieri

about 0.15 ml of extracted oil of Celestrus paniculatus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

When the Bacopa monnieri and Celestrus paniculatus oil were only administered, a significant nootropic was noticed in almost all the cases. The effect of hyper activity was minimum and the recall mechanism was also less.

### EXAMPLE X

A 10 ml composition in the form of syrup contained the following active ingredients:

about 200 mg of extract of Centella asiatica,

about 0.15 ml of oil of Celestrus paniculatus

and the balance amount is from known additives selected from stabilizers, anti-caking agents, flavoring agents, sweetening agents, thickening agents, preservatives and coloring agents.

Dosage:

| | |
|---|---|
| Children up to 5 years | about 5 ml-two times a day |
| Children above 5 years including adults | about 10 ml-2 times a day |

Dosages can be increased depending upon the severity of the disease or as prescribed by the doctor.

A significant psychotropic property was notices after continuous oral administration of the above composition. There was also improvement in the hyper activity but there was a minimum effect on recall mechanism.

### EXAMPLE XI

The Brahmi herb (Bacopa monnieri/Centella asiatica) as a complete plant was dried and extracted with absolute ethanol in a stainless steel pan. Four parts of ethanol was mixed with one part of herb and the material refluxed for about 10-12 hours. The process was repeated about 6 times till the herb is exhausted. All the above 6 extractions were concentrated to a paste of about 80% solids. This was done under vacuum and the solvents were recovered. The material was dried under reduced pressure at a temperature of about 50° C. in a vacuum drum drier. The powder obtained was extracted with pure chloroform (6 times) to remove the alkalides and oils. Identity test for alkaloids was performed. The material was filtered and chloroform insoluble fraction was concentrated to about 70% solids. The residue was dried in a vacuum drum drier. The dried powder was dissolved in about 6 times the quantity of distilled water and filtered. The supernatant was reduced to about 80% solids under vacuum and dissolved in pure Ethyl alcohol. The alcohol soluble fraction was reduced to about 80% solids under vacuum and dissolved in about 4 times the quantity of acetone. The acetone insoluble fraction was dried in drum drier. The dried powder was glycocidal rich fraction and debitterized extract of Brahmi herb. Identity test and quantitative analysis of saponins (Tritrepenic glycocides) was conducted. This fraction had saponin content of above about 15% to 20%.

In case of tablets, capsules or powder form, the Celestrus paniculatus oil is first adsorbed in Aerosol or any other suitable known adsorbing media and used accordingly.

If the composition is in the form of biscuits or snacks, each unit should contain the active ingredients as specified in the aforesaid examples.

Composition described and claimed is based on the herbal extracts in powder form and in the case Celestrus paniculatus in oil form. However, if the composition is made in liquid form, the extract described can also be used in paste/liquid form and the quantities calculated on the dry matter content corresponding to above composition.

Celestrus paniculatus oil is brought into water soluble phase by using Tween-20 or any such known emulsifying agent.

Conversion from ml. to mg.:

1 ml.×0.93=0.93 gm

0.93 gm×1000=930 mg

### INDUSTRIAL APPLICATION

The composition described above is useful for treating the diseases which cause the mental incapability's such as lower cholinergic activity, lower level of protein synthesis, presence of higher level of catecholamine, improper functioning of 5 HT in the brain, lower I.Q. level i.e. 70-50 and mental retardation caused by the following factors:

health of mother

genetic disease;

chromosomal diseases;

maternal illness;

anemia;

cardio-pulmonary diseases;

hemorrhage and hypertension;

malnutrition and the like.

We claim:

1. A highly potent herbal composition for improving overall mental performance in children, adults and mentally deficient people comprising a snynergistic combination of a hydro-alcoholic extract of Brahmi herb and at least one of a hydro-alcoholic extract of Acorus calamus and a Celestrus paniculatus oil.

2. A highly potent herbal composition for improving overall mental performance in children, adults and mentally deficient people comprising a hydro-alcoholic extract of Brahmi herb and at least one of a hydro-alcoholic extract of Acorus calamus and Celestrus paniculatus oil in the amounts as given below:

| | |
|---|---|
| Brahmi herb | about 100-500 mg |
| Acorus calamus | about 7.5-35 mg |
| Celestrus paniculatus oil | about 0.02-0.2 ml |

in a total volume of 10 ml or total weight of 11 gm, and the balance amount, if any, is from known additives selected from stabilizers, anti-caking agents, flavoring agents, sweetening agents, thickening agents, preservatives and coloring agents.

3. A herbal composition as claimed in claim 1 wherein the herbal extracts are in the form of a powder, liquid or paste and the quantities used are based on dry powder as given in the composition.

4. A herbal composition as claimed in claim 1 wherein Brahmi herb extract is either Bacopa monnieri or Centella asiatica or a combination of both.

5. A herbal composition as claimed in claim 4 wherein the preferred range of the following herbal material is as follows:

| | |
|---|---|
| Bacopa monnieri | about 150-300 mg |
| Centella asiatica | about 25-200 mg |
| Acorus calamus | about 8-20 mg |
| Celestrus paniculatus oil | about 0.04-0.15 ml |

6. A herbal composition as claimed in claim 4 wherein the Bacopa monnieri and Centella asiatica used in the composition have a saponin content about 15-25%.

7. A herbal composition as claimed in claim 1 wherein the Acorus calamus used has about 40-55% glycosides and β-asarone content below 5 ppm.

8. A herbal composition as claimed in claim 4 wherein Bacopa monnieri extract is an hydro-alcoholic extract.

9. A herbal composition as claimed in claim 4 wherein said Centella asiatica extract is an hydro-alcoholic extract.

10. A herbal composition as claimed in claim 5 wherein said Acorus calamus extract is hydro-alcoholic extract of the Rhizome of the herb and the extraction technique is adopted to either eliminate the β-asarone for obtaining extract of Acorus calamus with β-asarone less than 5 ppm wherein the content of β-asarone in the extract is measured by gas chromatography recommended by FDA, USA

11. A herbal composition as claimed in claim 1 wherein the Celestrus paniculatus is an oil.

12. A herbal composition as claimed in claim 4 wherein the extract of Bacopa monnieri for use in the composition is either prepared by fluxing a hydro-alcoholic extract of Bacopa monnieri, concentrating to a solid content of about 50-70% and drying the refluxed material at temperature about 50-90° C. under vacuum, treating the dried material with organic solvent preferably chloroform for removal of the soluble fraction and then subjecting the insoluble fraction to refining to obtain a debitterized rich fraction of glycosides.

13. A herbal composition as claimed in claim 1 wherein the said composition is in a powder, tablet, capsule, syrup or in suspension form and also is delivered through any other known carriers such as biscuits/snacks.

14. A herbal composition as claimed in claim 27 wherein a 10 ml composition in syrup form contains the following active ingredients:
about 120 mg of hydro-alcoholic extract of Bacopa monnieri
about 15 mg of hydro-alcoholic extract of Centella asiatica,
about 15 mg of β-asarone free hydro-alcoholic extract of Acorus calamus,
about 0.03 ml of Celestrus paniculatus oil
and the balance amount is from known additives selected from stabilizers, anti-caking agents, flavoring agents, sweetening agents, thickening agents, preservatives and coloring agents.

15. A herbal composition as claimed in claim 27 wherein a 10 ml composition in syrup form contains the following active ingredients:
about 250 mg of hydro-alcoholic extract of Bacopa monnieri
about 40 mg of hydro-alcoholic extract of Centella asiatica,
about 22 mg of β-asarone free-hydro-alcoholic extract of Acorus calamus,
about 0.15 ml of a Celestrus paniculatus oil
and the balance amount is from known additives selected from stabilizers, anti-caking agents, flavoring agents, sweetening agents, thickening agents, preservatives and coloring agents.

16. A herbal composition as claimed in claim 27 wherein a 10 ml composition in syrup form contains the following active ingredients:
about 250 mg of hydro-alcoholic extract of Bacopa monnieri
about 40 mg of hydro-alcoholic extract of Centella asiatica,
about 22 mg of β-asarone free hydro-alcoholic extract of Acorus calamus,
and the balance amount is from known additives selected from stabilizers, anti-caking agents, flavoring agents, sweetening agents, thickening agents, preservatives and coloring agents.

17. A herbal composition as claimed in claim 27 wherein a 10 ml composition in syrup form contains the following active ingredients:
about 200 mg of hydo-alcoholic extract of Bacopa monnieri
about 25 mg of β-asarone free hydro-alcoholic extract of Acorus calamus,
about 0.10 ml of Celestrus paniculatus oil
and the balance amount is from known additives selected from stabilizers, anti-caking agents, flavoring agents, sweetening agents, thickening agents, preservatives and coloring agents.

18. A herbal composition as claimed in claim 27 wherein a 10 ml composition in syrup form contains the following active ingredients:
about 200 mg of hydro-alcoholic extract of Bacopa monnieri
about 25 mg of β-asarone free hydro-alcoholic extract of Acorus calamus,
and the balance amount is from known additives selected from stabilizers, anti-caking agents, flavoring agents, sweetening agents, thickening agents, preservatives and coloring agents.

19. A herbal composition as claimed in claim 27 wherein a 10 ml composition in syrup form contains the following active ingredients:
about 200 mg of hydo-alcoholic extract of Centella asiatica,
about 25 mg of β-asarone free hydro-alcoholic extract of Acorus calamus,
about 0.10 ml of Celestrus paniculatus oil
and the balance amount is from known additives selected from stabilizers, anti-caking agents, flavoring agents, sweetening agents, thickening agents, preservatives and coloring agents.

20. A herbal composition as claimed in claim 27 wherein a 10 ml composition in syrup form contains the following active ingredients:
about 200 mg of hydro-alcoholic extract of Centella asiatica
about 25 mg of β-asarone free hydro-alcoholic extract of Acorus calamus
and the balance amount is from known additives selected from stabilizers, anti-caking agents, flavoring agents, sweetening agents, thickening agents, preservatives and coloring agents.

21. A herbal composition as claimed in claim 27 wherein a 10 ml composition in syrup form contains the following active ingredients:
about 200 mg of the hydro-alcoholic extract of Bacopa monnieri
about 40 mg of hydro-alcoholic extract of Centella asiatica,
about 0.10 ml of Celestrus paniculatus oil
and the balance amount is from known additives selected from stabilizers, anti-caking agents, flavoring agents, sweetening agents, thickening agents, preservatives and coloring agents.

22. A herbal composition as claimed in claim 27 wherein 10 ml composition in syrup form contains the following active ingredients:
about 200 mg of hydo-alcoholic extract of Bacopa nionnieri
about 0.15 ml of Celestrus paniculatus oil
and the balance amount is from known additives selected from stabilizers, anti-caking agents, flavoring agents, sweetening agents, thickening agents, preservatives and coloring agents.

23. A herbal composition as claimed in claim 27 wherein 10 ml composition in syrup form contains the following active ingredients:
about 200 mg of extract of Centella asiatica,
about 0.15 ml of Celestrus paniculatus oil
and the balance amount is from known additives selected from stabilizers, anti-caking agents, flavoring agents, sweetening agents, thickening agents, preservatives and coloring agents.

24. A herbal composition as claimed in claim 1 wherein when the composition is in the form of tablets, capsules or powder form, the Celestrus paniculatus oil is first adsorbed in Aerosol or any other suitable known adsorbing media and used accordingly.

25. A method for improving the mental ability of a mammal comprising administering an effective amount of the composition of claim 1.

26. The method of claim 25 wherein the mammal is a human.

27. A herbal composition as claimed in claimed in claim 2 wherein the Brahmi herb extract is either obtained from Bacopa monnieri or from Centella asiatica or from combination.
